SEQUENCE LISTING (13 sequences)

SEQ ID NO: 1 - DNA, 24 bp, Artificial Sequence
  Generated so that it is not found in the human genome
ctttatcaatacatactacaatca

SEQ ID NO: 2 - DNA, 24 bp, Artificial Sequence
  Generated so that it is not found in the human genome
tacactttatcaaatcttacaatc

SEQ ID NO: 3 - DNA, 24 bp, Artificial Sequence
  Generated so that it is not found in the human genome
caattcatttaccaatttaccaat

SEQ ID NO: 4 - DNA, 24 bp, Artificial Sequence
  Generated so that it is not found in the human genome
taatcttctatatcaacatcttac

SEQ ID NO: 5 - DNA, 24 bp, Artificial Sequence
  Generated so that it is not found in the human genome
tgattgtagtatgtattgataaag

SEQ ID NO: 6 - DNA, 24 bp, Artificial Sequence
  Generated so that it is not found in the human genome
gattgtaagatttgataaagtgta

SEQ ID NO: 7 - DNA, 24 bp, Artificial Sequence
  Generated so that it is not found in the human genome
attggtaaattggtaaatgaattg

SEQ ID NO: 8 - DNA, 24 bp, Artificial Sequence
  Generated so that it is not found in the human genome
gtaagatgttgatatagaagatta

SEQ ID NO: 9 - DNA, 49 bp, Artificial Sequence
  Generated so that it is not found in the human genome
gatacggaatcctaaggcatgggggttagtttctcgtgttccgtttgta

SEQ ID NO: 10 - DNA, 46 bp, Artificial Sequence
  Generated so that it is not found in the human genome
acgcatggactataataggatgaactcttctcgtgttccgtttgta

SEQ ID NO: 11 - DNA, 22 bp, Artificial Sequence
  Generated so that it is not found in the human genome
gagtacaaacggaacacgagaa

SEQ ID NO: 12 - DNA, 30 bp, Artificial Sequence
  Generated so that it is not found in the human genome
tataaaggaagccaaacttatttcccaggt

SEQ ID NO: 13 - DNA, 30 bp, Artificial Sequence
  Generated so that it is not found in the human genome
ttgacgcatggactataataggatgaactc

(19) United States
(12) Patent Application Publication
(10) Pub. No.: US 2011/0014609 A1
(43) Pub. Date: Jan. 20, 2011

(54) NUCLEIC ACID SIGNAL ENHANCEMENT USING NANOPARTICLE-BASED HYBRIDIZATION

(75) Inventors: Heather Newkirk, Overland Park, KS (US); Mauricio Miralles, Lee's Summit, MO (US)

Correspondence Address: POLSINELLI SHUGHART PC, 700 W. 47TH STREET, SUITE 1000, KANSAS CITY, MO, 64112-1802, US

(73) Assignee: THE CHILDREN'S MERCY HOSPITAL, Kansas City, MO (US)

(21) Appl. No.: 12/687,808

(22) Filed: Jan. 14, 2010

Related U.S. Application Data
(60) Provisional application No. 61/144,623, filed on Jan. 14, 2009.

(51) Int. Cl. C12Q 1/68

(52) U.S. Cl. 435/6

(57) ABSTRACT

A method of enhancing signal detection through use of nanoparticle-conjugated nucleic acid probes is provided. Following chromosomal FISH hybridization of a target sequence with a genomic probe linked to a flag sequence, the flag sequence is hybridized to an anti-flag sequence conjugated to a nanoparticle. The enhanced fluorescent probe is then visualized using microscopy.

### RELATED APPLICATION

This application claims the priority benefit of U.S. provisional patent application Ser. No. 61/144,623 filed on Jan. 14, 2009. The teachings and content of that application are hereby expressly incorporated by reference herein.

### BACKGROUND OF THE INVENTION

1. Field of the Invention

The present invention relates to a method of enhancing the signal detection of nucleic acid probes. The present invention further relates to a method of enhancing signal detection through use of nanoparticle-conjugated nucleic acid probes.

2. Description of the Prior Art

Use of nucleic acid probes to target homologous segments on target nucleic acid sequences is known in the prior art. Probes are typically labeled with a fluorochrome or other detectable moiety to allow probe detection using common methods such as FISH or flow cytometry. Detection, and in particular detection that is reproducible, can be complicated by low signal strength. Methods to boost signal strength include use of multiple probes, commonly referred to as a “cocktail”, selected to hybridize to a single target sequence so that each probe lends intensity to the overall signal strength of the probe. A disadvantage of this method is that it necessitates the identification of multiple target sites for hybridization and the preparation of multiple homologous probes.

The prior art does not teach or suggest a method using nanoparticle-coupled nucleic acid probes during FISH procedures. U.S. Pat. No. 6,280,946, to Hyldig-Nielsen et al., describes peptide nucleic acid (PNA) probes linked to nanospheres and employed in FISH experiments. These probes were not DNA probes, however, and the bacterial sequences detected were not in genomic DNA. In the present invention, non-human sequences are not used explicitly for the detection of a non-human target sequence. Rather, they are used as universal sequences, which are attached to nanoparticles. These non-human, nanoparticle-coupled, sequences are then hybridized to their reverse complement sequence linked to specific human genomic sequences, thus detecting human sequences using a non-human sequence as a bridge or intermediate. The use of a non-human sequence as an intermediate in FISH experiments for the detection of a specific human genomic sequence is not taught or suggested in the prior art.

Accordingly, what is needed in the art is a method for enhancing the signal corresponding to each probe to thereby reduce the number of probes required to generate a signal sufficient to allow the assay of a target sequence. Further, what is needed is a method of quickly identifying low copy segments that are thermodynamically suitable for hybridization from a known nucleic acid sequence of extended length.

### SUMMARY OF THE INVENTION

The present invention overcomes the problems inherent in the prior art and provides a distinct advance in the state of the art. The present invention provides a signal enhancing assembly and method of enhancing signal detection through use of nanoparticle-conjugated nucleic acid probes. The signal enhancing assembly of the present invention comprises a hybridization product that incorporates a nucleic acid probe, a first nucleic acid sequence, a second nucleic acid sequence, and nanoparticle. The first nucleic acid sequence and second nucleic acid sequence are also referred to as flag and anti-flag sequences (respectively). The flag and anti-flag sequences are complimentary to one another and, in preferred forms, the nanoparticle is attached to the anti-flag sequence. The flag and anti-flag sequences have no complementary sequence in the human genome. Preferably, the flag and anti-flag sequences are derived from an organism other than a human being.

The present invention also provides for a method of enhancing signal detection. The method comprises the steps of selecting an oligonucleotide probe capable of hybridizing to a target sequence; synthesizing said probe such that a first nucleic acid sequence, or flag sequence, is attached to the end of the probe; hybridizing said probe such that it binds to the target sequence; synthesizing a second nucleic acid sequence, or anti-flag sequence, complementary to the flag sequence, that is attached to a nanoparticle that emits a signal; hybridizing the flag sequence with the anti-flag sequence; and visualizing the hybridization signal produced.

The nanoparticle signal enhancement method of the present invention can be used for array CGH, microarray, FISH, QMH, or any other genomic hybridization experiment. The use of nanoparticles could help to identify sequences expressed at a low level in the genome, or could improve the overall signal on an array. Preferred nanoparticles include, but are not limited to particles, spheres, beads, and dendrimeres. Any nanoparticle that is capable of binding to a DNA sequence that can be used in a hybridization assay can be used for purposes of the present invention. In some preferred forms, a polymer bead or dendrimere is used. In a preferred embodiment carboxylated polystyrene nanoparticles (Invitrogen Corporation) are used. Preferred sizes for nanoparticles include, but are not limited to the following sizes, 1 nm, 40 nm, and 20 nm. The nanoparticles can be any color and multiple colors can be used when the invention is used to find and identify multiple targets. One common color of nanoparticles is red fluorescent (580 excitation/605 emission spectra). In a most preferred embodiment, a 20 nm nanoparticle is used. Several manufacturers produce color encoded polystyrene nanoparticles, any of which can be covalently linked to DNA by conventional chemistries. In the embodiment of the present invention incorporating a dendrimere, a fluroscent compound is incorporated to the dendrimere, such that the signal can be visualized using fluorescent technology. While any fluroscent compound will work, preferably, the compound is Texas Red, fluorescein, or a fluorescent nanoparticle.

In an alternate embodiment, the method of the present invention includes a nanoparticle or dendrimere and a conventional signal emitting moiety such as biotin, dig, or other biochemical side chain. In this embodiment, the probe is labeled with a signal emitting moiety and a flag sequence is attached to the probe and the anti-flag sequence having a nanoparticle or dendrimere attached thereto is hybridized to the flag sequence.

In a preferred embodiment, the probes used are single or low copy sequences. A single copy sequence is one that does not contain any repeat sequences and appears just one time in the haploid genome. A low copy sequence appears 10 or fewer times in the haploid genome. The probes can be of any length, and especially the lengths described in U.S. Pat. No. 7,014,997, the teachings and content of which are hereby incorporated by reference. Such probes are then fragmented by conventional techniques; preferably nick translation, sonication, or chemical or enzymatic methods. Preferably, the probes are fragmented such that the resulting fragments are from 50 bp to 1500 bp in length, more preferably, 60 bp to 1200 bp, even more preferably, 70 bp to 1000 bp, still more preferably 80 bp to 850 bp, even more preferably 90 bp to 800 bp, and still more preferably, 100 bp to 750 bp.

The present invention offers several advantages over the prior art. Fluorescent signals are at least 2 to 20 times more intense using the methods of the present invention. Additionally, the assay of the present invention is capable of utilizing universal flag and anti-flag sequences, thereby permitting many different genomic probes flagged with the same flag sequence to be combined in a single experiment and co-detected using the same anti-flag coupled nanoparticle.

The method according to the present invention can be used to detect and map genetic abnormalities. Genetic abnormalities include, but are not limited to, point mutations, SNPs, deletions, trinucleotide repeats, insertions, duplications, substitutions, frame shift mutations, and combinations thereof. In a preferred embodiment, the genetic abnormality is selected from the group consisting of point mutations, trinucleotide repeats, insertions, duplications, substitutions, and frame shift mutations. The genetic abnormalities can be associated with specific disease or cancer for diagnosis thereof, or can be used to determine a predisposition for a disease or cancer.

In a further embodiment of the present invention, a linker, preferably a carbon amine is attached to the probe. In this embodiment, the flag sequence is attached to the same end of the probe as the carbon amine. As is known in the art, a carbon amine can be of various carbon lengths and is preferably selected from a 6 carbon amine, a 12 carbon amine, an 18 carbon amine, a 24 carbon amine, a 30 carbon amine, or a 36 carbon amine. Preferably, the addition of a carbon amine provides for less steric interference when viewing the hybridization signal.

In another embodiment of the present invention, a signal enhancing assembly is provided. The signal enhancing assembly generally comprises a hybridization product comprising an oligonucleotide probe, a first nucleic acid sequence, a second nucleic acid sequence, and nanoparticle emitting a signal. Preferably, the first and second nucleic acid sequences are complementary to one another. A nanoparticle is attached to one of the first or second nucleic acid sequences. Preferably the first and second nucleic acid sequences have no complementary sequence in the human genome. In some forms of this embodiment, the first nucleic acid sequence is attached to the oligonucleotide probe. In other forms of this embodiment, the assembly further comprises a carbon amine attached to the probe on the same end as the first nucleic acid sequence. The carbon amine can be of any length, but preferably is selected from the group consisting of a 6 carbon amine, a 12 carbon amine, an 18 carbon amine, or a 24 carbon amine. In some forms of this embodiment, the oligonucleotide probe is a low copy probe. The nanoparticle can be any signal emitting moiety such as a nanoparticle. In some preferred forms, the signal emitting moiety is a bead or dendrimere.

In another embodiment of the present invention, a method of enhancing signal detection is provided. Generally, the method comprises the steps of:

i) selecting an oligonucleotide probe capable of hybridizing to a target sequence;

ii) attaching a first nucleic acid sequence to one end of said probe or synthesizing said probe such that a first nucleic acid sequence is attached to one end of said probe;

iii) hybridizing said probe such that it binds to the target sequence;

iv) synthesizing or obtaining a second nucleic acid sequence complementary to said first nucleic acid sequence;

v) attaching a signal emitting moiety to one of said first or second nucleic acid sequences;

vi) hybridizing said second nucleic acid sequence with said first nucleic acid sequence; and

vii) visualizing the hybridization signal emitted.

This embodiment can also include the step of attaching a linker, such as a carbon amine, to the probe on the same end of the probe as the first sequence. In preferred forms when using a carbon amine, the carbon amine is selected from the group consisting of a 6 carbon amine, a 12 carbon amine, an 18 carbon amine, or a 24 carbon amine. The oligonucleotide probe of this embodiment can be any sequence, but in some preferred forms is a low copy probe. The signal emitting moiety can be as described previously. Advantageously, this method can be performed in combination with FISH or QMH (quantum microsphere hybridization).

### DEFINITIONS

Unless defined otherwise, all technical and scientific terms used herein have the same meanings as commonly understood by one of ordinary skill in the art to which this invention belongs.

“Signal enhancing assembly” for purposes of the present invention, refers to the combination of a probe, a flag sequence, and an anti-flag sequence. In preferred forms, the assembly would further include a nanoparticle or dendrimere.

“Flag sequence” for purposes of the present invention, is a nucleic acid sequence that is not identical to sequence in the human genome and preferably is not capable of hybridizing with the human genome. In preferred forms, the flag sequence is derived from an organism other than a human being. In a preferred embodiment of the present invention, the flag sequence is attached to the 5′ end of the probe.

“Anti-flag sequence” for purposes of the present invention, is a nucleic acid sequence that is complementary to the flag sequence and is also not identical to a sequence in the human genome and preferably is not capable of hybridizing with the human genome. In preferred forms, the anti-flag sequence is attached to a nanoparticle, preferably a bead or dendrimere. Preferably, the anti-flag sequence is derived from an organism other than a human being.

A “nanoparticle” for purposes of the present invention is a particle having a diameter of from approximately 3 to approximately 100 nanometers, having any size, shape or morphology, and comprising a noble metal. The nanoparticle can be a particle, sphere, bead, dendrimere, nonodot or other particle customarily used in DNA nanotechnology. Preferably, the nanoparticle is a bead or dendrimere. For purposes of the present invention, a nanodot is a noble metal nanocluster that is encapsulated in an encapsulating material, such as a dendrimer or a peptide, wherein the encapsulated noble metal nanocluster is capable of fluorescing at a low excitation intensity.

An “enhanced signal” for purposes of the present invention is a signal that is at least two times brighter when visualized than those signals obtained using traditional hybridization and signal emitting methods. Preferably, an enhanced signal is at least 10 times brighter than those signals obtained using traditional hybridization and signal emitting methods.

### BRIEF DESCRIPTION OF THE DRAWINGS

FIG. 1 is a diagram showing signal enhancement achieved through using nanoparticle-conjugated anti-flag probes hybridized to flag probes previously hybridized to a target sequence;

FIG. 2 is a diagram showing signal enhancement including fluorochrome-labelled flag probes;

FIG. 3 is a photograph of a FISH slide showing nanoparticle-conjugated fluorescent probes visualized by microscopy and showing enhanced probe signal;

FIG. 4 is a diagram showing signal enhancement including single copy probes without the use of a nanoparticle; and

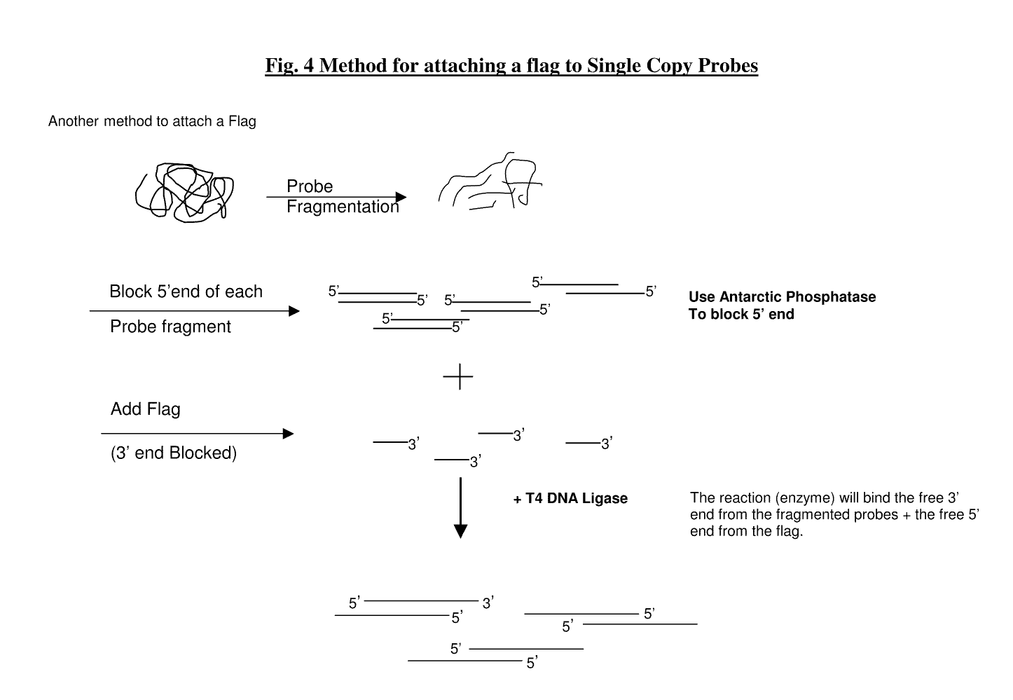

FIG. 5 is a diagram continuing the process described in FIG. 4 illustrating attaching nanoparticles to single copy probes.

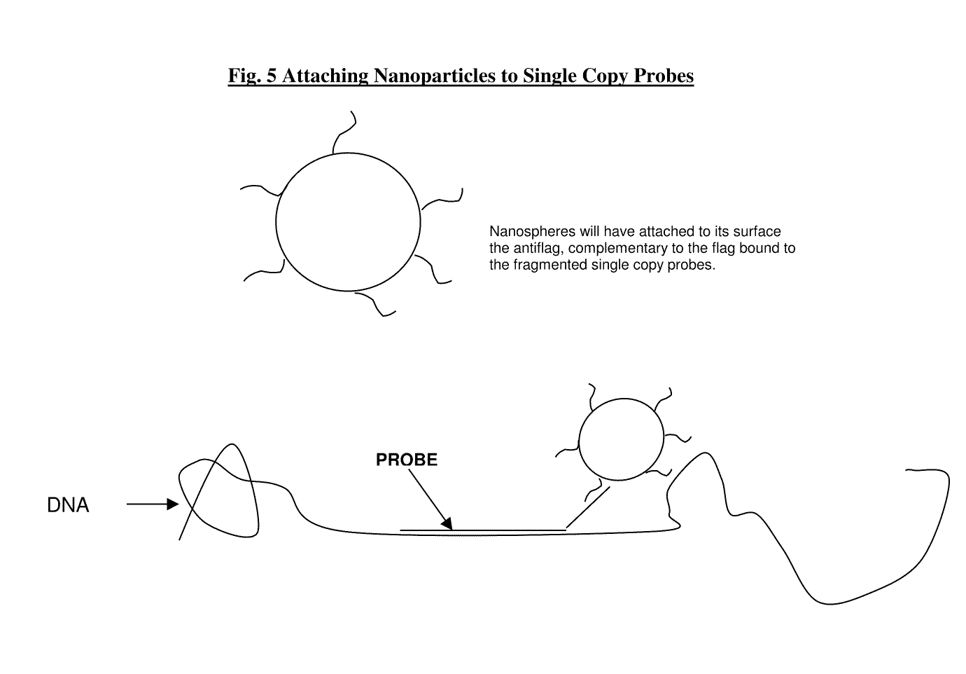

### DETAILED DESCRIPTION OF THE PREFERRED EMBODIMENTS

The following examples set forth preferred materials and procedures in accordance with the present invention. Although any methods and materials similar or equivalent to those described herein can be used in the practice or testing of the present invention, the preferred methods, devices, and materials are now described. It is to be understood, however, that these examples are provided by way of illustration only, and nothing therein should be deemed a limitation upon the overall scope of the invention.

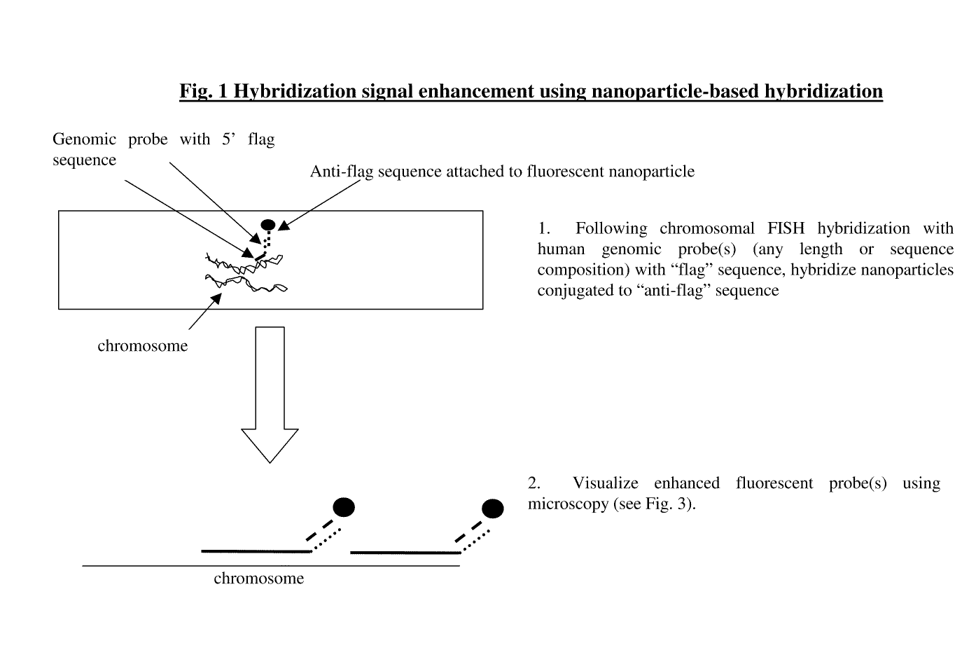

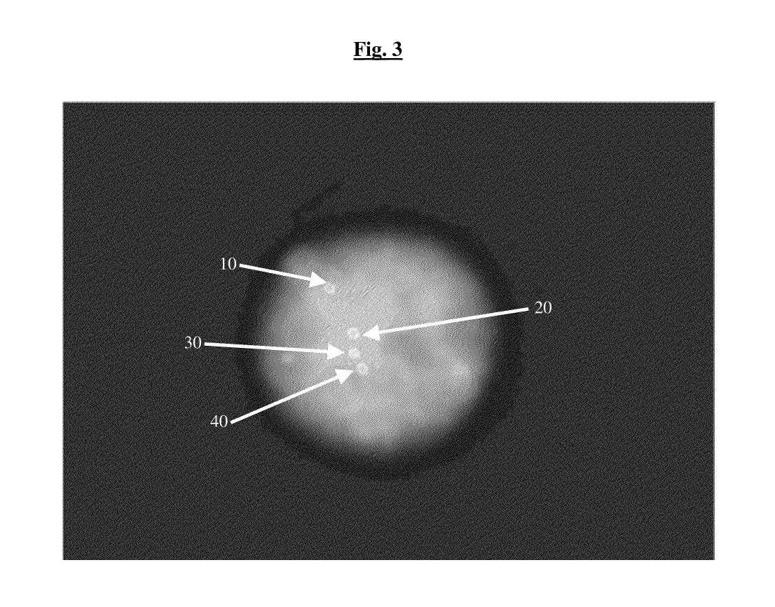

An embodiment of the present invention comprises a nanoparticle-based hybridization procedure for the enhanced detection of nucleic acid probes in genomic hybridization experiments, which offers an improvement to the existing method for in situ fluorescent hybridization (FISH), see FIG. 1. A 5′ amine-labeled nucleic acid probe containing sequence non-homologous to human genomic DNA (anti-flag) was conjugated via carbodiimide coupling to a suspension of carboxylated nanoparticles (<100 nm in diameter each). Sequence specific (not limited to single copy) human genomic probes were synthesized via PCR with a 5′ flag primer that is non-homologous to human genomic DNA but is complementary to the anti-flag sequence attached to the nanoparticle. In experiments, the PCR probe(s) ranged in length from 100 basepairs to 1000 basepairs. These genomic probes were then hybridized to fixed patient cells (methanol and acetic acid) immobilized on glass microscope slides using standard FISH techniques (Lichter et al. 2005). The anti-flag probe-conjugated nanoparticles were hybridized to the FISH slide and probes were visually detected by fluorescence microscopy. A photograph of a FISH slide showing nanoparticle-conjugated fluorescent probes 10, 20, 30, and 40 exhibiting enhanced probe signal is presented in FIG. 3. A single genomic probe with a flag sequence can be used, but for the best results, many genomic probes (>5) flagged with the same sequence were used in the FISH hybridization. Preferably, genomic probes are spaced approximately 10-20 base pairs apart in the genomic sequence to account for the steric hindrance of the hybridized nanoparticle.

The present invention has the advantage of providing brighter FISH probe signals. Further, the present invention allows for the enhancement of genomic probe signals using a layered approach wherein the genomic sequence is flagged with a sequence non-homologous to any genomic sequence. Subsequently, the complement to the flag sequence (anti-flag) attached to a nanoparticle is hybridized to the flag sequence in order to achieve a brighter fluorescent signal detected using a microscope. Since this flag sequence is not homologous to any human genomic sequence, there is no cross-hybridization of the anti-flag sequence conjugated to nanoparticles with any genomic sequence. The signal intensity of the probes of the present invention are preferably at least 2 times more intense than traditional fluorescent probes, even more preferably, at least 3 times more intense, more preferably, at least 4 times more intense, even more preferably, at least 5 times more intense, more preferably, at least 6 times more intense, more preferably, at least 7 times more intense, still more preferably, at least 8 times more intense, more preferably, at least 9 times more intense, even more preferably, at least 10 times more intense, and most preferably, at least 10.6 times more intense. In another embodiment, the signal intensity is 12-30 times more intense than a traditional fluorescent probe.

The present invention offers a further advantage of being compatable with traditional equipment used for FISH analysis. This assay developed according to the present invention can be used with standard equipment used for FISH analysis for detection of the nanoparticles. Further, no separate equipment or microscope filters are necessary.

In a preferred embodiment, the assay of the present invention utilizes a universal flag and anti-flag sequence, many different genomic probes flagged with the same flag sequence can be combined in a single experiment and co-detected using the same anti-flag coupled nanoparticle. In an alternate embodiment, nanoparticles with different spectral characteristics can be used to detect different genomic sequences.

### EXAMPLES

The assay is most useful for enhancement of FISH probe signals. However, other applications include, but are not limited to genomic hybridization experiments, such as array CGH, microarray, and quantitative microsphere hybridization (QMH).

### Example 1

### Materials and Methods

A sequence was selected that was thought to be non-complementary to human DNA sequences. After the sequence was selected, a BLAT search was completed to ensure that the sequence did not match any sequences within the human genome. Next, the flag/anti flag oligonucleotide sequences were obtained. One of the oligonucleotide flag sequences must have an amino linker to be able to attach it to the beads. Next, it was confirmed that the oligo attached to the bead (flag/ or antiFlag) did not cross-hybridize with genomic human DNA. At this point the Flag sequence was ready to use for further analysis. The flag/anti flag sequences used were as follows:

FLAG SEQUENCES: 5′->3′

| | |
|---|---|
| Flag 5: | |
| CTTTATCAATACATACTACAATCA | (SEQ ID NO: 1) |
| Flag 6: | |
| TACACTTTATCAAATCTTACAATC | (SEQ ID NO: 2) |
| Flag 7: | |
| CAATTCATTTACCAATTTACCAAT | (SEQ ID NO: 3) |
| Flag 8: | |
| TAATCTTCTATATCAACATCTTAC | (SEQ ID NO: 4) |

ANTIFLAG SEQUENCES: 5′->3′ (conjugate to beads)

| | |
|---|---|
| Antiflag 5: (5′ Amine)- | |
| TGATTGTAGTATGTATTGATAAAG | (SEQ ID NO: 5) |
| Antiflag 6: (5′ Amine)- | |
| GATTGTAAGATTTGATAAAGTGTA | (SEQ ID NO: 6) |
| Antiflag 7: (5′ Amine)- | |
| ATTGGTAAATTGGTAAATGAATTG | (SEQ ID NO: 7) |
| Antiflag 8: (5′ Amine)- | |
| GTAAGATGTTGATATAGAAGATTA | (SEQ ID NO: 8) |

Assays were developed and tested using genomic probes of varying lengths specific to ABL1 and HOXB1.

Results and Conclusions

There was no cross-hybridization with any of these flag sequences to the chromosomal DNA or labeled genomic probes. All probes showed brighter fluorescent signals when compared to the probes not flag-tagged and subsequently hybridized to anti-flag nanoparticles. The highest level of enhancement was seen with multiple (5-10) smaller probes (˜100 basepairs).

### Example 2

### Materials and Methods

This assay has also been tested using quantitative microsphere hybridization (QMH) as a genomic suspension hybridization platform. One hundred nucleotide ABL1 and HOXB1 probes were synthesized such that they each had a distinct non-human flag sequence and a carbon 6 amine at their 5′ end. These probes were conjugated to spectrally distinct microspheres (1.8 uM in diameter). Genomic DNA samples were biotinylated using a linear amplification method (GenomiPhi, GE Healthcare) modified by the addition of biotin 16-dUTP. The anti-flag sequences specific to each flag were synthesized with a 5′ C6 amine group, and conjugated to nanoparticles. The HOXB1 and ABL1 probes were hybridized in a multiplex reaction to each genomic DNA overnight at 50 C, and reactions were washed to remove all unhybridized genomic fragments.

Results and Conclusions

Probe signal intensities for ABL1 and HOXB1 increased by 10.6-fold when anti-flag conjugated microspheres were added as a second level of fluorescent detection as compared with those reactions without anti-flag conjugated microspheres. This ratio was directly calculated using mean fluorescence intensity values obtained via flow cytometry. Additionally, no genomic cross-hybridization was seen using the flag sequences attached to probes and anti-flag sequences conjugated to microspheres.

### Example 3

This example illustrates the use of dendrimeres as a signal enhancer for use with the probe system of the present invention utilizing QMH.

Materials and Methods

A capture probe is defined as a single copy probe complementary to a “unique” sequence in the human DNA, attached to a microsphere through a carbodiimide reaction. A reporter probe is a single or low copy probe complementary to a single or low copy sequence in the human DNA, with an “extra” non-human DNA sequence or flag attached at the 3′ or 5′ end thereof. The size of the flag can go from 10 to 30 bp. The flags used were between 20 and 30 bp. The distance between the capture probe and reporter probe should be long enough and not have any steric problem between the two probes; however the distance between them can not be extremely long either so the sequence where to reporter probe binds is deleted, translocated.

A capture probe and reporter probe were created as described below.

Capture Probe: IC1A Reporter: IC1b Antiflag
Sequence:
(SEQ ID NO: 12)
IC1A 5′-TATAAAGGAAGCCAAACTTATTTCCCAGGT
(SEQ ID NO: 9)
IC1b Antiflag 5′-
GATACGGAATCCTAAGGCATGGGGGTTAGTTTCTCGTGTTCCGTTTGTA
Capture Probe: HoxBlb Reporter: HoxB1a Antiflag
Sequence:
(SEQ ID NO: 13)
HoxB1 5′-TTGACGCATGGACTATAATAGGATGAACTC
(SEQ ID NO: 10)
HoxBla Antiflag 5′-
ACGCATGGACTATAATAGGATGAACTCTTCTCGTGTTCCGTTTGTA
(SEQ ID NO: 11)
Dendrimer FLAG
5′-GAGTACAAACGGAACACGAGAA
*Underlined sequences represent the complementary
sequences between Flag/Antiflag probes
Non-biotinilated DNA was used as a template for
the capture and reporter probes. The capture
probes IC1A and HoxB1 and reporter probes IC1b
and HoxBla were used for the assay.

The master mix was prepared as follows:

| | | 9.5X (Master Mix) |
|---|---|---|
| 1.5X TMAC | 45 μl | 427.5 μl |
| DNA | 1 μl | X |
| IC1A | 1 μl | 9.5 μl |
| IC1b Antiflag | 1 μl | 9.5 μl |
| HoxB1b | 1 μl | 9.5 μl |
| HoxB1a Antiflag | 1 μl | 9.5 μl |
| | ~50 μl | |

The master mix was prepared and vortexed. Then 49 ul of master mix was added to each tube. Next, 1 ul of human non-biotinylated DNA (50 ng·ul) was added to each tube. The tubes were then incubated at 95° C. for 5 min and then hybridized for three (3) hours using a gradient temperature between 58-68° C. After incubation, 150 ul of 1×TMAC was added to each reaction tube and mixed very well. The reaction tubes were then centrifuged at 16,000×g for 2 minutes. After centrifugation, the supernate containing the non-captured DNA was discarded. Next, 2.5 μl of dendrimer (UltraAmp Capture Sequence Cap03/Biotin Label (350)) and 50 μl 1×TMAC were added to the reaction. The reaction tubes were then incubated at 50° C. for 30 minutes. Next, each reaction tube was washed with 250 ul of 1×TMAC and centrifuged at 16,000×g for 2 minutes. The supernate was carefully removed from each reaction. Next, 12.5 ul of SPE (1 mg/ml) was added at a 1:50 dilution in 1×TMAC. The reaction tubes were then incubated at 50° C. for 10 minutes. After incubation, 250 ul of 1×TMAC was added to each tube and vortexed well. The tubes were centrifuged at 16,000×g for 2 minutes. Again, the supernate was removed. The pellet was then resuspended in 70 ul of 1×TMAC and analyzed.

Results and Conclusions

TABLE 1
Results of hybridization

| | | | |
|---|---|---|---|
| Temp | IC1A | HoxB1b | IC1A/HoxB1b |

| | | |
|---|---|---|
| ° C. | MFI | ratio |

| | | | |
|---|---|---|---|
| 58.0 | 711 | 765 | 0.93 |
| 58.9 | 705 | 729 | 0.97 |
| 59.7 | 693 | 757 | 0.92 |
| 60.8 | 695 | 717 | 0.97 |
| 62.3 | 677 | 723 | 0.94 |
| 64.0 | 649 | 687 | 0.94 |
| 65.4 | 645 | 663 | 0.97 |
| 67.3 | 621 | 605 | 1.03 |

Since normal human DNA was used in the experiment, a 1.0 MFI ratio between Hoxb1b (internal control) and IC1A is expected. Column 4 from Table 1 shows the expected ratio between the two probes. The results show that under the conditions specified in the protocol, the Capture/Reporter Dendrimer QMH works.

### Example 4

This example will illustrate the use of dendrimeres with FISH analysis using a biotinylated probe.

Materials and Methods

### Probe Preparation

The sequences for the probes will be selected and amplified by PCR. The amplified product will be purified by Agarose gel followed by gel extraction. The probes will then be nick-translated with 16-biotin-dUTP. The nick-translation probes will then be purified by Ethanol precipitation. The probes will then be resuspended in ddH2O or Elution Buffer (EB, 10 mM Tris-HCl, pH 7.4) to a final concentration of 100 ng/ul.

### Slide Pretreatment

Slides are obtained and incubated in 2×SSC at 42° C. (in coplin jar) for 10 min. Then the slides are incubated in 70%, 80%, 90% and 100% ethanol at room temperature (RT) for 1 min each. The slides are then allowed to air dry. Next, the slides are incubated in 2×SSC/70% formamide at 72° C. for 3 min and then incubated twice in cold 70% ethanol for 1 minute each time. Then the slides are incubated in 80%, 90%, and 100% ethanol at room temperature for 1 minute each and allowed to air dry.

Blocking in situ Biotin

A solution is prepared with an antibiotin antibody (1 mg/ml) at a 1:500 dilution in 4×SCC, 1% BSA at 37° C. for 1 hour. The slides are then washed twice with a 4×SSC, 1% BSA solution for 5 minutes at room temperature.

### Hybridization and Denaturation

### DAY1:

The hybridization solution is pre-warmed at 37° C. A tube is prepared containing a DNA probe (˜200 ng) and 10 ul dd formamide is added. The tube is then incubated at 72° C. for 5 minutes, then placed on ice for at least 5 minutes. Next, 10 ul of Hybridization solution is added. The solution in the tube was mixed well and spun down for 30 seconds at 13000×g. Then 20 ul of solution is added to the slide. The tube is covered with a plastic coverslip and parafilm is used to make a closed chamber to maintain humidity. The covered tube is then incubated at 37° C. overnight.

### DAY 2:

Post-Hybridization Wash

| | | | |
|---|---|---|---|
| 1^{st} wash: | 25 mL regular formamide | | |
| | 5 mL 20X SSC | | |
| | 20 mL dH₂O | | |
| | 50 mL | → | Prewarm to 42° C. |
| | | | Slide is washed for |
| | | | 20 min and shaken. |
| 2^{nd} wash: | 5 mL 20X SSC | | |
| | 45 mL dH₂O | | |
| | 50 mL | → | Prewarm to 42° C. |
| | | | Slide is washed for |
| | | | 20 min and shaken. |
| 3^{rd} wash: | 2.5 mL 20X SSC | | |
| | 47.5 mL dH₂O | | |
| | 50 mL | → | Room temperature. |
| | | | Slide is washed for |
| | | | 20 min and shaken. |

### Detection

The dendrimer used is Strepavidin-oyster 660.

10 ng (1 ul) of streptavidin-ouster 660 is diluted in 40 ul of hybridization buffer. Then 40 ul of detection buffer is added. The marked area on the slide is then covered with parafilm and the slide is incubated for 1 hour at 37° C. in the dark.

### Post-Detection Wash

The slides are washed in 40% formamide/2×SSC for 5 minutes at 37° C. in an aluminum foil covered coplin jar and shaken occasionally. Next, the slides are washed in 2×SSC for 10 minutes at room temperature in aluminum covered coplin jar and shaken.

### Counterstain

The slides are then counterstained by the following procedure:

50 ul DAPI (0.2 ug/mL) is added to the center of the slide. The slide is then covered with parafilm to make a parafilm sandwich. The slide is then incubated for 7 to 10 minutes at room temperature in the dark. The slides are then rinsed for 5 min in McIlvaines Buffer for DAPI pH 7.0. The McIlvaines Buffer is prepared using Citric Acid H₃C₆H₅O₇) (1.26 g) (ACROS lot A009817601); Sodium Phosphate Dibasic (Na₂HPO₄) (12.38 g)(SIGMA lot 53H0264); ddH₂O (fill to just under 1000 mL); 6N HCl to adjust pH to 7.0; and fill to 1000 mL. Alternately, PBS may be used together with VectaShield with Dapi.

### Mount Slides w/ Coverslips

6 ul mounting media (anti-fade) is added to the slides, which are then covered with glass coverslips (no fingerprints). The bubbles, if any, are removed by gently tapping the slide. The edges of the slide are then sealed with clear nail polish and stored in a light tight black box at 20° C.

### Results and Conclusions

The results will show that the dendrimere is activated when it hybridized thereby producing a brighter fluorescent signal than traditional FISH probes.

### Example 5

This example illustrates the use of dendrimeres with FISH analysis using a non-biotinylated probe.

### Materials and Methods

The sequences for the probes will be selected and amplified by PCR. The amplification will incorporate primers or attach a flag sequence to the 3′ end. The amplified product will be purified by Agarose gel electrophoresis followed by gel extraction. The probes will then be resuspended in ddH2O or Elution Buffer (EB, 10 mM Tris-HCl, pH 7.4) to a 100 ng/ul.

### Slide Pretreatment

Slides are obtained and incubated in 2×SSC at 42° C. (in coplin jar) for 10 min. Then the slides are incubated progressively in 70%, 80%, 90% and 100% ethanol at room temperature (RT) for 1 minute each. The slides are then allowed to air dry. Next, the slides are incubated in 2×SSC/70% formamide at 72° C. for 3 minutes and then incubated twice in cold 70% ethanol for 1 minute each time. Then the slides are then incubated in 80%, 90%, and 100% ethanol at room temperature for 1 minute each and then allowed to air dry.

### Hybridization and Denaturation

### DAY1:

The hybridization solution is pre-warmed at 37° C. A tube is prepared containing a DNA probe (˜2 uL) and 10 ul dd formamide is added. The tube is incubated at 72° C. for 5 minutes, then placed on ice for at least 5 minutes. Next, 10 ul of hybridization solution is added. The solution in the tube was mixed well and spun down for 30 seconds at 13,000×g. Then 20 ul of solution is placed on the slide. The tube is covered with a plastic coverslip and parafilm is used to seal the chamber to maintain humidity inside. The covered tube is then incubated at 37° C. overnight.

### DAY 2:

Post-Hybridization Wash

| | | | |
|---|---|---|---|
| 1^{st} wash: | 25 mL regular formamide | | |
| | 5 mL 20X SSC | | |
| | 20 mL dH₂O | | |
| | 50 mL | → | Prewarm to 42° C. |
| | | | Slide is washed for |
| | | | 20 min and shaken. |
| 2^{nd} wash: | 5 mL 20X SSC | | |
| | 45 mL dH₂O | | |
| | 50 mL | → | Prewarm to 42° C. |
| | | | Slide is washed for |
| | | | 20 min and shaken. |
| 3^{rd} wash: | 2.5 mL 20X SSC | | |
| | 47.5 mL dH₂O | | |
| | 50 mL | → | Room temperature. |
| | | | Slide is washed for |
| | | | 20 min and shaken |

### Detection

The dendrimer used is Strepavidin-oyster 660. The dendrimere used will have the anti-flag sequence attached, which is complimentary to the flag sequence.

10 ng (1 ul) of streptavidin-ouster 660 is diluted in 40 ul of hybridization buffer. Then 40 ul of detection buffer is added. The marked area on the slide is covered with parafilm followed by incubation for 1 hour at 37° C. in the dark.

### Post-Detection Wash

The slides are washed in 40% formamide/2×SSC for 5 minutes at 37 C in an aluminum foil covered coplin jar and shaken occasionally. Next, the slides are washed in 2×SSC for 10 minutes at room temperature in an aluminum foil covered coplin jar and shaken.

### Counterstain

The slides are then counterstained by the following procedure:

50 ul DAPI (0.2 ug/mL) is added to the center of the slide. The slide is then covered with parafilm to make a parafilm sandwich. The slide is then incubated for 7 to 10 minutes at room temperature in the dark. The slides are then rinsed for 5 minutes in Mcllvaines Buffer for DAPI pH 7.0. Alternately, PBS may be used as well along with VectaShield with Dapi.

Mount Slides w/Coverslips

6 ul mounting media (anti-fade) is added to the slides, which are then covered with glass coverslip (no fingerprints). The bubbles, if any, are removed by gently tapping the slide. The edges of the slide are then sealed with clear nail polish and stored in a light tight black box at −20° C.

### Results and Conclusions

The results will show that the dendrimere is activated when it hybridized thereby producing a brighter fluroscent signal than traditional FISH probes.

### Example 6

The incorporation of a flag sequence to a 3′ end can be used with single copy probes, or non-single copy probes (i.e BACs, cosmids, etc), double or single stranded DNA, RNA, etc. This methodology ensures that every fragment created has a flag sequence attached thereto.

### Materials and Methods

This Example provides an alternative protocol for attaching a first oligonucleotide sequence (i.e., a flag sequence) to the 3′ end of an oligonucleotide probe. This protocol can be used with single copy oligonucleotide probes (sequences that appear only one time in the human genome), low copy oligonucleotide probes (sequences that appear 10 or fewer times in the human genome), other non-low or non-single copy probes (i.e. BACs, cosmids, ½ BACS, YACS, etc.), double stranded DNA, RNA, and the like.

To begin, a pure oligonuceotide probe is fragmented by either physical (i.e. sonication) or chemical, or enzymatic (i.e. DNAse). If an enzymatic procedure is selected, the DNA fragments are preferably cleaned by EtOH precipitation, or using a column purification protocol, such as the QIAquick PCR Purification Kit.

Next the 5′ end of the probe is blocked with Antarctic Phosphatase by adding 1/10 volume (5 μl) of 10× Antarctic Phosphatase (New England Biolabs) to 1 ug of DNA probe. The volume is then adjusted to 50 μl with ddH20 to which 1 ul of Antarctic phosphatase (5 units) is added and mixed. This mixture is incubated for 15 or 60 minutes at 37 C, whether the oligonucleotide fragments have 5′ extension or blunt-ends, or 3′ extensions. The reaction is then heat inactivated at 65 C for 5 minutes before proceeding with ligation.

To ligate, use T4 DNA Ligase with FLAG (3′ end blocked) and add 1/10 volume (7.5 μl) of T4 DNA ligase buffer. The final volume is adjusted to 75 ul with sterile ddH2O and 400 units of T4 DNA ligase is added for every 20 ul reaction volume. This is then incubated at room temperature for 2 hours and subsequently heat inactivated at 65 C for 10 minutes. The flag label probe can then be stored at −20 C. This probe would then be ready for use with the normal nano-FISH protocol. The second oligonucleotide sequence (i.e., the antiflag sequence) would be attached to the nanosphere as described herein.

### Results

The results will show a brighter, more easily visualizable, fluorescent signal, when hybridized, than traditional FISH probes.

### Example 7

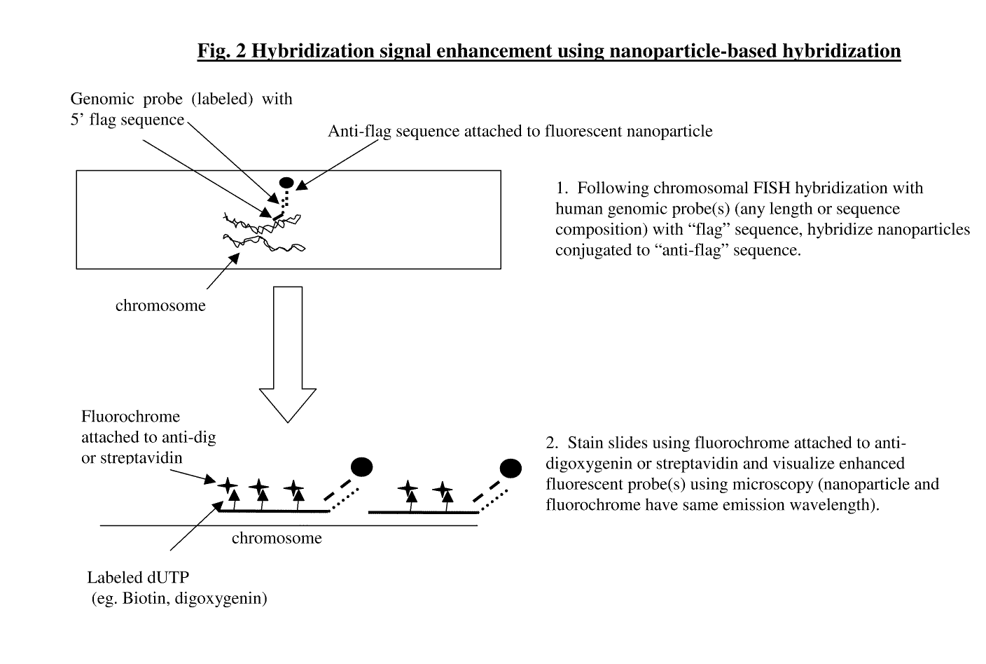

In an alternative embodiment of the invention (see FIG. 2), during PCR synthesis, biotin dUTP or digoxygenin dUTP was incorporated into the probe(s). These labeled genomic probes were then hybridized to fixed human cells (methanol and acetic acid) immobilized on glass microscope slides using standard FISH techniques (Lichter et al. 2005). The anti-flag probe-conjugated nanoparticles were hybridized to the FISH slide and subsequently stained with a streptavidin conjugate or anti-digoxygenin fluoroscein. Probes were visually detected by fluorescence microscopy. The emission wavelength of the antibody-conjugated fluorochrome and the internal spectral signature of the nanosphere were optimized to be similar if not identical to produce the greatest enhancement of probe signal.

1. A signal enhancing assembly comprising a hybridization product comprising an oligonucleotide probe, a first nucleic acid sequence, a second nucleic acid sequence, and a signal emitting moiety, said first and second nucleic acid sequences being complementary to one another, wherein said signal emitting moiety is attached to one of said first or second nucleic acid sequences and wherein said first and second nucleic acid sequences have no complementary sequence in the human genome.

2. The assembly of claim 1, where said first nucleic acid sequence is attached to said oligonucleotide probe.

3. The assembly of claim 2, further comprising a carbon amine attached to the probe on the same end as said first nucleic acid sequence.

4. The assembly of claim 3, wherein said carbon amine is selected from the group consisting of a 6 carbon amine, a 12 carbon amine, an 18 carbon amine, or a 24 carbon amine.

5. The assembly of claim 3, wherein said oligonucleotide probe is a low copy probe.

6. The assembly of claim 1, wherein said signal emitting moiety is a nanoparticle.

7. The assembly of claim 1, wherein said nanoparticle is a bead or dendrimere.

8. A method of enhancing signal detection comprising the following steps:
i) selecting an oligonucleotide probe capable of hybridizing to a target sequence;
ii) attaching a first nucleic acid sequence to one end of said probe or synthesizing said probe such that a first nucleic acid sequence is attached to one end of said probe;
iii) hybridizing said probe such that it binds to the target sequence;
iv) synthesizing or obtaining a second nucleic acid sequence complementary to said first nucleic acid sequence;
v) attaching a signal emitting moiety to one of said first or second nucleic acid sequences;
vi) hybridizing said second nucleic acid sequence with said first nucleic acid sequence; and
vii) visualizing the hybridization signal emitted.

9. The method of claim 8, further comprising a carbon amine attached to said probe on the same end of the probe as said first sequence.

10. The method of claim 9, wherein said carbon amine is selected from the group consisting of a 6 carbon amine, a 12 carbon amine, an 18 carbon amine, or a 24 carbon amine.

11. The method of claim 8, wherein said oligonucleotide probe is a low copy probe.

12. The method of claim 8, wherein said signal emitting moiety is a nanoparticle.

13. The method of claim 8, wherein said enhanced signal detection is used in combination with FISH.

14. The method of claim 8, wherein said enhanced signal detection is used in combination with QMH.

15. A method of enhancing signal detection comprising the following steps:
i) selecting an oligonucleotide probe capable of hybridizing to a target sequence;
ii) attaching a first nucleic acid sequence to one end of said oligonucleotide probe or synthesizing said oligonucleotide probe such that a first nucleic acid sequence is attached to one end of said oligonucleotide probe;
iii) attaching a linker to one of said first or second nucleotide sequences
iv) hybridizing said probe such that it binds to the target sequence;
v) synthesizing or obtaining a second nucleic acid sequence complimentary to said first nucleic acid sequence;
vi) attaching a signal emitting moiety to one of said first or second nucleic acid sequences;
vii) hybridizing said second nucleic acid sequence with said first nucleic acid sequence; and
viii) visualizing the hybridization signal emitted.

16. The method of claim 15 wherein said linker is a carbon amine.

17. The method of claim 16, wherein said carbon amine is selected from the group consisting of a 6 carbon amine, a 12 carbon amine, an 18 carbon amine, or a 24 carbon amine.

18. The method of claim 15, wherein said oligonucleotide probe is a low copy probe.

19. The method of claim 15, wherein said enhanced signal detection is used in combination with FISH.

20. The method of claim 15, wherein said enhanced signal detection is used in combination with QMH.
